(19) United States
(12) Patent Application Publication
(10) Pub. No.: US 2024/0357984 A1
(43) Pub. Date: Oct. 31, 2024

(54) METHOD FOR ENHANCING INDUCTION EFFICIENCY OF CORN HAPLOID

(71) Applicant: Institute of Food Crops, Yunnan Academy of Agricultural Sciences, Kunming (CN)

(72) Inventors: Xingming Fan, Kunming (CN); Xingfu Yin, Kunming (CN); Fuyan Jiang, Kunming (CN); Yaqi Bi, Kunming (CN)

(21) Appl. No.: 18/764,248

(22) Filed: Jul. 4, 2024

(30) Foreign Application Priority Data
Jul. 7, 2023 (CN) 202310830371.1

(51) Int. Cl. A01H 1/02; A01H 6/46

(52) U.S. Cl. CPC A01H 1/02; A01H 6/4684

(57) ABSTRACT

A method for enhancing induction efficiency of corn haploid is disclosed, which belongs to the technical field of genetics. In the method disclosed by the disclosure, the tropical induction line is used as a female parent, the temperate induction line is used as a male parent, hybrid seeds among the temperate and tropical induction lines are generated by hybridizing and assembling, the hybrid vigor among the temperate and tropical induction is used for improving the agronomy and yield traits of the conventional induction line, so that the requirements of the corn haploid induction line as the male parent on plant height, pollen quantity, fertility period and the like are met, which has important significance for large-scale popularization and application of the corn double haploid (DH) technology.

### TECHNICAL FIELD

The disclosure relates to the technical field of genetics, in particular to a method for enhancing induction efficiency of corn haploid.

### BACKGROUND ART

The traditional corn inbred line breeding needs 8-10 generations of continuous inbreeding, which takes 4-5 years, with a long breeding cycle and high costs, and the technology of double haploid (DH) of corn only needs 2 steps of utilizing a corn haploid induction line as a male parent to carry out hybridization induction on female parent materials to generate haploids, and obtaining a homozygous inbred line in a doubling mode, so that the breeding time of the corn inbred line is shortened to 1-2 years, the breeding efficiency can be greatly improved, and the breeding cost is reduced.

However, the existing corn haploid induction lines in China are almost temperate induction lines at present, for example, No. 5 (CAU5) selected and developed by the China Agricultural University exhibits the features of poor adaptability, small pollen volume, poor setting characteristic, short plant, difficulty in propagation and the like in the tropical and subtropical regions, and cannot be directly utilized by the tropical and subtropical regions; the tropical corn haploid induction line shows outstanding advantages in disease resistance, pollen quantity and adaptability, for example, Yun induction No. 2 selected and developed by the food crop institute of the agricultural sciences of Yunnan province has outstanding resistance to diseases such as gray spot disease, white spot disease, rust disease and the like, has good self-setting characteristic, but has a long growth period, and for the large-scale application of the corn haploid induction technology by using the induction line as a male parent, the plant height, pollen quantity, growth period and the like of the induction line cannot meet the requirements of the male parent of the seed production induction line. Based on the above, it is urgently required to select the warm-heat combined induction line combination with strong adaptability, high induction rate, high plant height, large pollen quantity and moderate growth period for corn haploid breeding and scientific research.

Accordingly, it would be desirable to provide a method for enhancing induction efficiency of corn haploid.

### SUMMARY

In view of the above, the present disclosure provides a method for enhancing induction efficiency of corn haploid.

The utilization of hybrid vigor has become a common method for improving crop yield and adaptability in crop breeding and production. The present disclosure uses the tropical induction line (such as Yun induction No. 2) with strong disease resistance and good setting characteristic as the female parent, the temperate induction line as the male parent (such as CAU5) to hybridize and assemble the hybrid seeds among the warm induction lines, and uses the hybrid advantage of the induction line hybrid combination F₁ in terms of induction rate, pollen quantity, seed set rate, disease resistance and plant height to realize the requirements of large-scale application of corn haploid induction technology in the manner of “seed production” for the induction line as the male parent.

In order to achieve the above purpose, the present disclosure adopts the following technical scheme:

A method for enhancing induction efficiency of corn haploid includes the following specific steps:
(1) using a tropical induction line as a female parent, using a temperate induction line as a male parent, generating hybrid seeds among the temperate and tropical induction lines by hybridizing and assembling to obtain an induction line hybrid combination F₁;
(2) using the induction line hybrid combination F₁ as a male parent, using a material to be induced as a female parent, performing a haploid induction in a manner of “seed production”; pulling out male flowers of the female parent line with one to two leaves in time when female parent tasseling, so that pollens of the induction line hybrid combination F₁ naturally disperse; regulating a sowing interval of the male parent and the female parent according to characteristics of flowering time of both parties;
(3) harvesting separately according to the parent materials, and writing a label notice board;
(4) carrying out large-scale selection on haploids according to a traditional corn haploid induction line R1-nj genetic marker color developing principle after female parent ears are harvested and aired, and selecting grains with endosperm color development and embryo non-color development as quasi-haploid grains;
(5) performing field planting and natural doubling on the obtained quasi-haploid grains, and performing bagging self-pollination to obtain successfully doubled corn double haploid ears; and
(6) carrying out threshing and seedling transplanting on each successfully doubled corn double haploid ear, carrying out transplanting on seedlings at a three-leaf stage according to an ear-row method, carrying out fine management in the field, carrying out target character evaluation on the ears in a whole growth period, carrying out bagging and selfing seed reserving, and determining final selection of double haploid ear rows according to the target character.

Further, the tropical induction line is Yun induction No. 2.

Further, the temperate induction line is CAU5.

Further, the material to be induced is Yunrui No. 6, YML107, YML46, YML16.

Further, in step (2), the haploid induction in a manner of “seed production” is carried out by using a specification of 1:4-6 of male parent and female parent.

Further, in step (2), the haploid induction in a manner of “seed production” is carried out by using a specification of 1:5 of male parent and female parent.

Further, in step (2), that a specific operation of regulating a sowing interval of the male parent and the female parent according to characteristics of flowering time of both parties is as follows: a sowing time of the first-stage male parent of is regulated according to a difference in days between the female parent's silking and the male parent's pollen dispersal, the second-stage male parent and the female parent are sown at the same time, then a stage of male parent is sowed every 3 days, and the male parent is sowed for a total of 3-4 stages.

Further, the fine management in step (6) includes: watering, weeding, fertilizing and insecticides.

Further, the target character in step (6) includes: good disease resistance, strong adaptability and high self-yield.

Compared with the prior art, the method for enhancing induction efficiency of corn haploid is disclosed, the tropical induction line is used as a female parent, the temperate induction line is used as a male parent, hybrid seeds among the temperate and tropical induction lines are generated by hybridizing and assembling, the hybrid vigor among the temperate and tropical induction is used for improving the agronomy and yield traits of the conventional induction line, so that the requirements of the corn haploid induction line as the male parent on plant height, pollen quantity, fertility period and the like are met, the technical problem that neither temperate nor tropical corn haploid induction lines can meet the requirements of induction rate, pollen quantity, seed setting rate, disease resistance, plant height and the like on male parent induction line when corn haploid induction technology is applied on a large scale in a manner of “seed production”, and the method has important significance on large-scale engineering popularization and application of the corn double haploid technology.

### DETAILED DESCRIPTION OF THE EMBODIMENTS

Technical solutions in the embodiments of the present disclosure will be clearly and completely described below. Obviously, the described embodiments are only part of the embodiments of the present disclosure, not all of them. Based on the embodiments of the disclosure, all other embodiments made by those skilled in the art without sparing any creative effort should fall within the protection scope of the disclosure.

The disclosure uses the tropical induction line (such as Yun induction No. 2) with strong disease resistance and good setting characteristic as the female parent and the temperate induction line (such as CAU5) as the male parent to generate hybrid seeds among the temperate and tropical induction lines, and utilize the hybrid vigor of the induction line hybrid combination F₁ (Yun induction No. 2×CAU5) on induction rate, pollen quantity, seed setting rate, disease resistance, plant height and the like to meet the requirements on the induction line as a male parent when corn haploid induction technology is applied on a large scale in a manner of “seed production”.

### Embodiment 1

A method for enhancing induction efficiency of corn haploid includes the following specific steps:

The tropical induction line (such as Yun induction No. 2) with strong disease resistance and good setting characteristic is used as a female parent, and the temperate induction line (such as CAU5) is used as a male parent to generate hybrid seeds among the temperate and tropical induction lines by hybridizing and assembling, so that the induction line hybrid combination F₁ (Yun induction No. 2×CAU5) is obtained, and the seed quantity of F₁ depends on the actual induction scale.

(2) The induction line hybrid combination F₁ (Yun induction No. 2×CAU5) is used as male parent, the material to be induced (Yunrui No. 6, YML107, YML46, YML 16) is used as a female parent, a haploid induction in a manner of “seed production” is performed by using a specification of 1:5 of male parent and female parent, that is, the male flowers of the female parent line with one to two leaves are pulled out in time when female parent tasseling, so that pollens of the induction line hybrid combination F₁ (Yun induction No. 2×CAU5) naturally disperse. The whole operation process is like the conventional seed production in the field, the artificial supplementary pollination is not needed, the method is convenient and quick, the induction rate is high, and the yield is high. And the sowing interval of the male parent and female parent can be regulated according to the characteristics of flowering time of both parties: the silking of the female parent Yunrui No. 6, YML107, YML46 and YML16 is respectively 5 days earlier, 3 days earlier, 7 days later and 5 days later compared with the pollen dispersing of the male parent, and the regulation is carried out by comprehensively considering the difference in days between the female parent's silking and the male parent's pollen dispersal: early sowing the first-stage male parent, sowing the second-stage male parent and all female parents after 5 days, and sowing the a stage of male parent every 3 days, wherein the male parent is sowed for 4 stages altogether so as to ensure that the flowering periods of the male parent and the female parent meet fully;

(3) When harvesting, harvesting is carried out separately according to the parent material, and a label notice board is written;

(4) After female parent ears are harvested and aired for 4-5 days, large-scale selection on haploids is carried out according to a traditional corn haploid induction line R1-nj genetic marker color developing principle, that is, grains with endosperm color development and embryo non-color development are selected as quasi-haploid grains;

(5) Field planting and natural doubling are performed on the obtained quasi-haploid grains, and bagging self-pollination is performed to obtain successfully doubled corn double haploid (DH line) cars;

(6) Threshing and seedling transplanting are carried out on each successfully doubled corn double haploid (DH line) car, transplanting is carried out on seedlings at a three-leaf stage according to an car-row method, fine management (watering, weeding, fertilizing and insecticides) is carried out in the field, target character (good disease resistance, strong adaptability and high self-yield) evaluation is carried out on the cars in a whole growth period, bagging and selfing seed reserving are carried out, and final selection of double haploid (DH) car rows is determined according to the target character.

### Embodiment 2

Experiments were performed according to the embodiment 1 in Jinghong city (winter) and Kunming city (spring) in Yunnan province in 2020-2022 with a planting density of 4500 plants/mu, and the experimental results are shown in tables 1-2. The improvement was specifically carried out in Jinghong city in winter and in Kunming city in spring alternately, and the improvement was continuous operation in one year.

TABLE 1
Specific implementation steps of a method for
enhancing induction efficiency of corn haploid

| | | Implementation |
|---|---|---|
| Material Genealogy | Method | time |
| Yun induction No. 2 | As a female parent | Winter 2020 |
| CAU5 | As a male parent | Winter 2020 |
| Yun induction No. 2 × CAU5 | Obtaining F₁ through | Winter 2020 |
| | hybridization | |
| Different materials to be | As the female parent, | Spring 2021 |
| induced 1-4 (1-4 refers | removing one to two | |
| to four different breeding | leaves from the male | |
| materials to be induced: | flowers of the female | |
| Yunrui 6, YML107, YML46, | parent row | |
| YML16 | | |
| Yun induction No. 2 × CAU5 | Planting in the male | Spring 2021 |
| | parent row in a 1:5 ratio, | |
| | naturally dispersing | |
| | powder | |
| (Different materials to be | Obtaining first filial | Spring 2021 |
| induced 1-4) × (Yun | generation through | |
| induction No. 2 × CAU5) | hybridization | |
| Haploid (different materials | Carrying out selection | Spring 2021 |
| to be induced 1-4) × (Yun | on haploids according to | |
| induction No. 2 × CAU5) | an induction line R1-nj | |
| | genetic marker color | |
| | developing principle | |
| DH [(different induced | Naturally doubling to | Winter 2020 |
| materials 1-4) × (Yun | obtain double haploid | |
| induction No. 2 × CAU5)] | (DH line) ears | |
| DH [(different induced | Plant identification of | Spring 2021 |
| materials 1-4) × (Yun | double haploid (DH | |
| induction No. 2 × CAU5)] | line) using the ear-row | |
| | method according to the | |
| | standard in step (6) | |

TABLE 2
Induction rate effect comparison of methods for
enhancing induction efficiency of corn haploid

| | | | | | | Improvement | |
|---|---|---|---|---|---|---|---|
| | | | | | | effect of | |
| | | | | | | induction | |
| | | | | | | rate (%) on | |
| | | | | | | Yun | |
| | | Induction | | | Average | induction | |
| Material | Pollen | setting | Disease | Plant | induction | No. 2 × | |
| Genealogy | quantity | rate | resistance | height | rate (%) | CAU5 | Time |
| (Different | medium | Medium | Good | Medium | 15.0 | 10 | Spring |
| materials to | | | | | | | 2021 |
| be induced | | | | | | | |
| 1-4) × Yun | | | | | | | |
| induction No. 2 | | | | | | | |
| (CK1) | | | | | | | |
| (Different | Small | poor | poor | short | 7.5 | 120 | Spring |
| materials to | | | | | | | 2021 |
| be induced | | | | | | | |
| 1-4) × CAU5 | | | | | | | |
| (CK2) | | | | | | | |
| (Different | large | Good | Good | high | 16.5 | / | Spring |
| materials to | | | | | | | 2021 |
| be induced | | | | | | | |
| 1-4) × (Yun | | | | | | | |
| induction No. | | | | | | | |
| 2 × CAU5) | | | | | | | |

The induction rate calculation formula: Number of haploid plants identified in the field/total number of grains×100%

The average induction rate calculation formula: sum of induction rates of different materials to be induced/number of materials to be inducted

As can be seen from Table 2, compared with the two parents of the original induction lines (Yun induction No. 2 and CAU5), the hybrid combination of the induction lines (Yun induction No. 2×CAU5) shows hybrid vigor in terms of pollen quantity, induction setting rate, disease resistance, plant height, average induction rate and the like, which shows that (Yun induction No. 2×CAU5) is more suitable for the requirements of male parent induction lines when corn haploid induction technology is used on a large scale in a manner of “seed production”.

The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

What is claimed is:

1. A method for enhancing induction efficiency of corn haploid, comprising following specific steps:
(1) using a tropical induction line as a female parent, using a temperate induction line as a male parent, generating hybrid seeds among the temperate and tropical induction lines by hybridizing and assembling to obtain an induction line hybrid combination F₁;
(2) using the induction line hybrid combination F₁ as a male parent, using a material to be induced as a female parent, performing a haploid induction in a manner of “seed production”; pulling out male flowers of the female parent line with one to two leaves in time when female parent tasseling, so that pollens of the induction line hybrid combination F₁ naturally disperse; regulating a sowing interval of the male parent and the female parent according to characteristics of flowering time of both parties;
(3) harvesting separately according to the parent materials, and writing a label notice board;
(4) carrying out large-scale selection on haploids according to a traditional corn haploid induction line R1-nj genetic marker color developing principle after female parent ears are harvested and aired, and selecting grains with endosperm color development and embryo non-color development as quasi-haploid grains;
(5) performing field planting and natural doubling on the obtained quasi-haploid grains, and performing bagging self-pollination to obtain successfully doubled corn double haploid ears; and
(6) carrying out threshing and seedling transplanting on each successfully doubled corn double haploid ear, carrying out transplanting on seedlings at a three-leaf stage according to an ear-row method, carrying out fine management in the field, carrying out target character evaluation on the ears in a whole growth period, carrying out bagging and selfing seed reserving, and determining final selection of double haploid ear rows according to the target character.

2. The method of claim 1, wherein the tropical induction line is Yun induction No. 2.

3. The method of claim 1, wherein the temperate induction line is CAU5.

4. The method of claim 1, wherein the material to be induced is Yunrui No. 6, YML107, YML46, YML16.

5. The method of claim 1, wherein in step (2), the haploid induction in a manner of “seed production” is carried out by using a specification of 1:4-6 of male parent and female parent.

6. The method of claim 5, wherein in step (2), the haploid induction in a manner of “seed production” is carried out by using a specification of 1:5 of male parent and female parent.

7. The method of claim 1, wherein in step (2), that a specific operation of regulating a sowing interval of the male parent and the female parent according to characteristics of flowering time of both parties is as follows: a sowing time of the first-stage male parent of is regulated according to a difference in days between the female parent's silking and the male parent's pollen dispersal, the second-stage male parent and the female parent are sown at the same time, then a stage of male parent is sowed every 3 days, and the male parent is sowed for a total of 3-4 stages.

8. The method of claim 1, wherein the fine management in step (6) comprises: watering, weeding, fertilizing and insecticides.

9. The method of claim 1, wherein the target character in step (6) comprises: good disease resistance, strong adaptability and high self-yield.
